(19) United States
(12) Patent Application Publication
(10) Pub. No.: US 2025/0073018 A1
(43) Pub. Date: Mar. 6, 2025

(54) OPHTHALMIC DEVICES HAVING A HIGH REFRACTIVE INDEX

(71) Applicant: Bausch + Lomb Ireland Limited, Dublin (IE)

(72) Inventors: Walter Laredo, Fort Worth, TX (US); Borui Zhang, Rochester, NY (US); Jeffrey Linhardt, San Francisco, CA (US)

(21) Appl. No.: 18/774,161

(22) Filed: Jul. 16, 2024

Related U.S. Application Data
(60) Provisional application No. 63/535,368, filed on Aug. 30, 2023.

(51) Int. Cl. A61F 2/16

(52) U.S. Cl. CPC A61F 2/16; A61F 2240/001

(57) ABSTRACT

An ophthalmic device which is a polymerization product of a monomeric mixture including (a) one or more heterocyclic monomers having an ethylenically unsaturated reactive end group, (b) one or more aromatic monomers having an ethylenically unsaturated reactive end group, and (c) one or more crosslinking agents, wherein the ophthalmic device has a refractive index of greater than or equal to about 1.50.

### PRIORITY CLAIM

The present application claims priority to U.S. Provisional Patent Application Ser. No. 63/535,368, entitled “Ophthalmic Devices Having a High Refractive Index,” filed Aug. 30, 2023, the content of which is incorporated by reference herein in its entirety.

### BACKGROUND

One of the more common human eye disorders involves progressive clouding of the natural crystalline lens matrix resulting in the formation of what is referred to as a cataract. It is now common practice to cure a cataract by surgically removing the clouded, natural lens and replacing it with an artificial intraocular lens (IOL) in a surgical procedure known as cataract extraction. In the extracapsular extraction method, the natural lens is removed from the lenticular capsular bag while leaving the posterior part of the capsular bag and preferably at least part of the anterior part of the capsular bag in place within the eye. In this instance, the lenticular capsular bag remains anchored to the eye's ciliary body through the zonular fibers. The capsular bag also continues its function of providing a natural barrier between the aqueous humor at the front of the eye and the vitreous humor at the rear of the eye.

### SUMMARY

In accordance with an illustrative embodiment, an ophthalmic device is a polymerization product of a monomeric mixture comprising:
(a) one or more heterocyclic monomers having an ethylenically unsaturated reactive end group,
(b) one or more aromatic monomers having an ethylenically unsaturated reactive end group, and
(c) one or more crosslinking agents,
wherein the ophthalmic device has a refractive index of greater than or equal to about 1.50.

In accordance with another illustrative embodiment, a method for making an ophthalmic device comprises:
(a) providing a monomeric mixture comprising (i) one or more heterocyclic monomers having an ethylenically unsaturated reactive end group, (ii) one or more aromatic monomers having an ethylenically unsaturated reactive end group, and (iii) one or more crosslinking agents, and
(b) subjecting the monomeric mixture to polymerizing conditions to provide an ophthalmic device having a refractive index of greater than or equal to about 1.50.

### DETAILED DESCRIPTION

Various illustrative embodiments described herein are directed to ophthalmic devices such as intraocular lenses having a high refractive index. As mentioned above, one of the more common human eye disorders involves progressive clouding of the natural crystalline lens matrix resulting in the formation of what is referred to as a cataract. It is now common practice to cure a cataract by surgically removing the cataractous human crystalline lens (i.e., a natural lens) and implanting an artificial intraocular lens in the eye to replace the natural lens.

In general, hydrogel materials have a relatively low refractive index, making them less desirable than other materials because of the thicker lens optic necessary to achieve a given refractive power. Silicone materials generally have a higher refractive index than hydrogels, but tend to unfold explosively after being placed in the eye in a folded position. Explosive unfolding can potentially damage the corneal endothelium and/or rupture the natural lens capsule. Acrylic materials are desirable because they typically have a higher refractive index than silicone materials and unfold more slowly or controllably than silicone materials.

Materials that are used to replace the natural crystalline lens must be soft and have excellent flexibility so that, once formed into a lens, they may be folded and passed through an incision which is typically about 2 millimeters (mm). Furthermore, the material must have excellent transparency and little to no glistening. Having a high refractive index allows for a thinner lens to be used. A material with a high Abbe number demonstrates less dispersion. This, in turn, allows for improved optical results and less light scattering. Thus, there is a need for combining a high refractive index with a high Abbe number to provide desired optical characteristics for a material.

Accordingly, the ophthalmic devices described herein overcome the foregoing problems and advantageously provide an ophthalmic device having a high refractive index. In some embodiments, the ophthalmic devices described herein overcome the foregoing problems and advantageously provide an ophthalmic device having a high refractive index and a high Abbe number. In non-limiting illustrative embodiments, the ophthalmic devices described herein are a polymerization product of a monomeric mixture comprising (a) one or more heterocyclic monomers having an ethylenically unsaturated reactive end group, (b) one or more aromatic monomers having an ethylenically unsaturated reactive end group, and (c) one or more crosslinking agents.

In an illustrative embodiment, as may be combined with one or more of the preceding paragraphs, the ophthalmic devices described herein have a refractive index of greater than or equal to about 1.50. In another illustrative embodiment, the ophthalmic devices described herein have a refractive index of greater than or equal to about 1.50 to about 1.55. In another illustrative embodiment, the ophthalmic devices described herein have a refractive index of about 1.51 to about 1.55.

In one or more additional non-limiting illustrative embodiments, as may be combined with one or more of the preceding paragraphs, the ophthalmic devices described herein have an Abbe number greater than or equal to 38. In one or more additional non-limiting illustrative embodiments, the ophthalmic devices described herein have an Abbe number greater than or equal to 38 to about 55. In one or more additional non-limiting illustrative embodiments, the ophthalmic devices described herein have an Abbe number greater than or equal to 40 to about 55. In the foregoing illustrative embodiments, an upper limit for the Abbe number can be less than or equal to 60.

Refractive index and Abbe Number are both measured using an Abbe refractometer and a material sample that has been equilibrated in balanced salt solution at 35° C. prior to measurement.

Refractive index is defined as:

N=c/v

where c is the speed of light in a vacuum and v is the phase velocity of light in the medium.

The “Abbe number,” also known as the V-number or constringence of a transparent material, is a measure of the material's dispersion, i.e., variation of refractive index versus wavelength, with high values of V indicating low dispersion. The Abbe number (v_{D}) is calculated using the following formula:

$${V}_{D} = \frac{{n}_{D} - 1}{{n}_{F} - {n}_{C}}$$

where n_{D}, n_{F}, and n_{C} are the refractive indices of the material at the wavelengths of 589 nanometers (nm) (sodium D), 486 nm (hydrogen F), and 656 nm (hydrogen C), respectively.

In one or more additional non-limiting illustrative embodiments, as may be combined with one or more of the preceding paragraphs, the glass-transition temperature (“Tg”) of the ophthalmic devices described herein, which affects the ophthalmic device's folding and unfolding characteristics, is at an upper limit of less than or equal to about 30° C., or less than or equal to about 27° C., or less than or equal to about 25° C., or less than or equal to about 20° C., or less than or equal to about 15° C., or less than or equal to about 12° C., or less than or equal to about 10° C. and a lower limit of about 6° C., or about 8° C., or about 10° C., or about 15° C., where any of the lower limits can be combined with any of the upper limits.

In one or more additional non-limiting illustrative embodiments, as may be combined with one or more of the preceding paragraphs, the Tg of the ophthalmic devices can be less than or equal to about 30° C. in the dry state, and less than or equal to about 20° C. in the hydrated state.

Tg is measured by differential scanning calorimetry at 5° C./minute, and is determined as the half-height of the heat capacity increase.

In one or more additional non-limiting illustrative embodiments, as may be combined with one or more of the preceding paragraphs, the ophthalmic devices described herein can have an equilibrium water content (EWC) of less than or equal to about 10%. In one or more additional non-limiting illustrative embodiments, as may be combined with one or more of the preceding paragraphs, the ophthalmic devices described herein can have an equilibrium water content (EWC) of greater than about 1% and up to about 10%.

In non-limiting illustrative embodiments, as may be combined with one or more of the preceding paragraphs, component (a) of the monomeric mixture comprises one or more heterocyclic monomers having an ethylenically unsaturated reactive end group. In an illustrative embodiment, the heterocyclic monomers having an ethylenically unsaturated reactive end group can be present in the monomeric mixture in an amount ranging from about 20 weight percent to about 75 weight percent, based on the total weight of the monomeric mixture. In an illustrative embodiment, the heterocyclic monomers having an ethylenically unsaturated reactive end group can be present in the monomeric mixture in an amount ranging from about 20 weight percent to about 50 weight percent, based on the total weight of the monomeric mixture.

Ethylenically unsaturated reactive end groups are well known to those skilled in the art. Suitable ethylenically unsaturated polymerizable groups include, for example, (meth)acrylates, vinyl carbonates, O-vinyl carbamates, N-vinyl carbamates, styrene-containing radicals and (meth)acrylamides. As used herein, the term “(meth)” denotes an optional methyl substituent. Thus, terms such as “(meth)acrylate” denotes either methacrylate or acrylate, and “(meth)acrylamide” denotes either methacrylamide or acrylamide. In one embodiment, an ethylenically unsaturated reactive end group can be represented by the general formula:

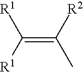

wherein R¹ is independently hydrogen, fluorine or methyl; R² is independently hydrogen, fluorine, an alkyl radical having 1 to 6 carbon atoms, or a —CO—Y—R³ radical wherein Y is —O—, —S— or —NH— and R³ is a divalent alkylene radical having 1 to about 10 carbon atoms.

In another illustrative embodiment, the (meth)acrylic group of a heterocyclic (meth)acrylic monomer is a (meth)acrylate-containing reactive end group. Suitable (meth)acrylate-containing reactive end groups can be those represented by the structure:

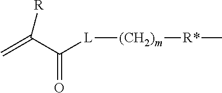

wherein R is hydrogen or methyl; L is O, NR¹, or S, where R¹ is H, CH₃, CH₂CH₃, or CH(CH₃)₂; m is an integer from 0 to 4 and R* is a linking group or bond. Suitable linking groups include, for example, any divalent hydrocarbon radical or moiety such as independently straight or branched, substituted or unsubstituted C₁-C₆ alkyl group, or an —OR² group where R² is an alkyl group from 1 to 6 carbon atoms.

The term “heterocyclic,” by itself or in combination with other terms, means, unless otherwise stated, a non-aromatic cyclic version of “alkyl” and “heteroalkyl,” respectively, wherein the carbons making up the ring or rings do not necessarily need to be bonded to a hydrogen due to all carbon valencies participating in bonds with non-hydrogen atoms. Representative examples of heterocyclic ring groups of the heterocyclic monomer having an ethylenically unsaturated reactive end group for use herein include, by way of example, a substituted or unsubstituted stable 3 to about 15 membered ring radical, containing carbon atoms and from one to five heteroatoms, e.g., nitrogen, phosphorus, oxygen, sulfur and mixtures thereof. Suitable heterocyclic ring radicals of the heterocyclic monomer having an ethylenically unsaturated reactive end group for use herein may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states. Examples of such heterocyclic ring radicals include, but are not limited to, azetidinyl, carbazolyl, dioxolanyl, piperidinyl, piperazinyl, pyrrolidinyl, 2-oxopiperazinyl, 2-oxopiperidinyl, 2-oxopyrrolidinyl, morpholinyl, and the like and mixtures thereof.

In an illustrative embodiment, a heterocyclic moiety may include one ring heteroatom (e.g., O, N, S or P). In an illustrative embodiment, a heterocycloalkyl moiety may include two optionally different ring heteroatoms (e.g., O, N, S, Si, or P).

The heterocyclic monomers having an ethylenically unsaturated reactive end group can be unsubstituted or substituted. In an illustrative embodiment, a substituted heterocyclic monomer having an ethylenically unsaturated reactive end group include the heterocyclic ring portion of the heterocyclic monomer being substituted with one or more of a hydroxy group, halogen group, carboxyl group, cyano group, nitro group, oxo (═O) group, thio (═S) group, a substituted or unsubstituted alkyl group, or a substituted or unsubstituted alkoxy group.

In a non-limiting illustrative embodiment, a heterocyclic (meth)acrylic monomer is represented by a structure of Formula I:

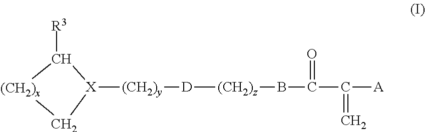

wherein x is an integer from 1 to 6, or from 1 to 5, or from 1 to 4 or from 1 to 2; y is an integer from 0 to 3 or from 1 to 3; R³ is hydrogen, hydroxy group, halogen group, carboxyl group, cyano group, nitro group, oxo (═O) group, thio (═S) group, a substituted or unsubstituted alkyl group, or a substituted or unsubstituted alkoxy group; X is a heteroatom such as O, N, S or P; B is O, NR, or S, where R is H, CH₃, CH₂CH₃, or CH(CH₃)₂; D is O, S, or a bond; A is H or CH₃; and z is 0 to 4, provided that if D is a bond then at least one of y and z is 0.

In an illustrative embodiment, the one or more heterocyclic (meth)acrylic monomers are represented by a structure of Formula I, wherein x is an integer from 1 to 4; y is 0; R³ is an oxo group, X is N; D is O; z is an integer from 1 to 4; B is O; and A is H or CH₃.

In an illustrative embodiment, the one or more heterocyclic (meth)acrylic monomers are represented by a structure of Formula I, wherein x is an integer from 1 to 3; y is 0; R³ is an oxo group, X is N; D is O; z is an integer from 2 to 3; B is O; and A is H or CH₃.

In an illustrative embodiment, the one or more cycloaliphatic (meth)acrylic monomers are represented by a structure of Formula I, wherein x is an integer from 1 or 2; y is an integer from 1 to 4; R³ is an oxo group, X is N, D is a bond; z is 0; B is O; and A is H or CH₃.

In an illustrative embodiment, the one or more heterocyclic (meth)acrylic monomers include, for example, 3-(2-oxopyrrolidin-1-yl)propyl acrylate, 2-(2-oxopyrrolidin-1-yl)ethyl acrylate, 3-(2-oxopyrrolidin-1-yl)propyl methacrylate, 2-(2-oxopyrrolidin-1-yl)ethyl methacrylate, (2-oxopyrrolidin-1-yl)methyl methacrylate, 4-(2-oxopyrrolidin-1-yl)butyl methacrylate, (2-oxopyrrolidin-1-yl)methyl acrylate, 4-(2-oxopyrrolidin-1-yl)butyl acrylate, 3-(1H-pyrrol-1-yl)propyl acrylate), 3-(1H-pyrrol-1-yl)propyl methacrylate and the like and mixtures thereof.

In non-limiting illustrative embodiments, as may be combined with one or more of the preceding paragraphs, component (b) of the monomeric mixture comprises one or more aromatic monomers having an ethylenically unsaturated reactive end group. In an illustrative embodiment, the aromatic monomers having an ethylenically unsaturated reactive end group can be present in the monomeric mixture in an amount of less than or equal to about 80 weight percent, based on the total weight of the monomeric mixture. In an illustrative embodiment, the aromatic monomers having an ethylenically unsaturated reactive end group can be present in the monomeric mixture in an amount of about 30 weight percent to about 80 weight percent, based on the total weight of the monomeric mixture. In an illustrative embodiment, the aromatic monomers having an ethylenically unsaturated reactive end group can be present in the monomeric mixture in an amount ranging from about 40 weight percent to about 80 weight percent, based on the total weight of the monomeric mixture.

The term “aromatic” as used herein refers to a conjugated cyclic unsaturated hydrocarbon typified by the benzene skeleton, the naphthalene skeleton or the like in a molecule. Representative examples of aromatic groups of the aromatic monomers having an ethylenically unsaturated reactive end group for use herein include, by way of example, a substituted or unsubstituted monoaromatic or polyaromatic radical containing from about 5 to about 25 carbon atoms or from 6 to 12 carbon atoms or from 6 to 10 carbon atoms such as, for example, phenyl, naphthyl, tetrahydronapthyl, indenyl, biphenyl and the like, optionally containing one or more heteroatoms, e.g., O and N, and the like.

In non-limiting illustrative embodiments, as may be combined with one or more of the preceding paragraphs, the aromatic monomers having an ethylenically unsaturated reactive end group can be one or more arylalkyl monomers having an ethylenically unsaturated reactive end group.

Representative examples of arylalkyl groups for use herein include, by way of example, a substituted or unsubstituted aromatic group as defined above directly bonded to an alkyl group, e.g., —CH₂C₆H₅, —C₂H₅C₆H₅ and the like.

Representative examples of alkyl groups for use herein include, by way of example, a straight or branched hydrocarbon chain radical containing carbon and hydrogen atoms of from 1 to about 18 carbon atoms or from 1 to 6 carbon atoms with or without unsaturation, to the rest of the molecule, e.g., methyl, ethyl, n-propyl, 1-methylethyl (isopropyl), n-butyl, n-pentyl, etc., and the like.

The ethylenically unsaturated reactive end group of the aromatic monomers having an ethylenically unsaturated reactive end group can be any of those discussed above. In an illustrative embodiment, an ethylenically unsaturated reactive end group is a (meth)acrylate end group.

Specific examples thereof of an aromatic (meth)acrylic monomer include 2-phenylethyl acrylate, 2-phenylethyl methacrylate, 2-phenoxyethyl acrylate, 2-phenoxycthyl methacrylate, 3-phenylpropyl acrylate, 3-phenylpropyl methacrylate and the like and mixtures thereof.

In non-limiting illustrative embodiments, as may be combined with one or more of the preceding paragraphs, component (c) of the monomeric mixture comprises one or more crosslinking agents. Suitable crosslinking agents for use herein are known in the art. For example, in non-limiting illustrative embodiments, suitable one or more cross-linking agents include one or more crosslinking agents containing at least two ethylenically unsaturated reactive end groups. In one embodiment, the ethylenically unsaturated reactive end groups are (meth)acrylate-containing reactive end groups. In another embodiment, the ethylenically unsaturated reactive end groups are non-(meth)acrylate reactive end groups. In one embodiment, the ethylenically unsaturated reactive end groups are a combination of one or more (meth)acrylate-containing reactive end groups and one or more non-(meth)acrylate reactive end groups.

In an illustrative embodiment, useful one or more crosslinking agents containing at least two ethylenically unsaturated reactive end groups include, for example, one or more di-, tri- or tetra(meth)acrylate-containing crosslinking agents. In an illustrative embodiment, useful one or more di-, tri- or tetra(meth)acrylate-containing crosslinking agents include, for example, alkanepolyol di-, tri- or tetra(meth)acrylate-containing crosslinking agents such as, for example, one or more alkylene glycol di(meth)acrylate crosslinking agents, one or more alkylene glycol tri(meth)acrylate crosslinking agents, one or more alkylene glycol tetra(meth)acrylate crosslinking agents, one or more alkanediol di(meth)acrylate crosslinking agents, alkanediol tri(meth)acrylate crosslinking agents, alkanediol tetra(meth)acrylate crosslinking agents, agents, one or more alkanetriol di(meth)acrylate crosslinking agents, alkanetriol tri(meth)acrylate crosslinking agents, alkanetriol tetra(meth)acrylate crosslinking agents, agents, one or more alkanetetraol di(meth)acrylate crosslinking agents, alkanetetraol tri(meth)acrylate crosslinking agents, alkanetetraol tetra(meth)acrylate crosslinking agents and the like and mixtures thereof.

In an illustrative embodiment, one or more alkylene glycol di(meth)acrylate crosslinking agents include tetraethylene glycol dimethacrylate, ethylene glycol di(meth)acrylates having up to about 10 ethylene glycol repeating units, butyleneglycol di(meth)acrylate and the like. In one embodiment, one or more alkanediol di(meth)acrylate crosslinking agents include butanediol di(meth)acrylate crosslinking agents, hexanediol di(meth)acrylate and the like. In one embodiment, one or more alkanetriol tri(meth)acrylate crosslinking agents are trimethylol propane trimethacrylate crosslinking agents. In one embodiment, one or more alkanetetraol tetra(meth)acrylate crosslinking agents are pentaerythritol tetramethacrylate crosslinking agents.

In a non-limiting illustrative embodiment, suitable crosslinking agents include, for example, ethylene glycol diacrylate, diethylene glycol diacrylate, allyl acrylate, 1,3-propanediol diacrylate, 2,3-propanediol diacrylate, 1,6-hexanediol diacrylate, 1,4-butanediol diacrylate, tricthylene glycol diacrylate, cyclohexane-1,1-diyldimethanol diacrylate, 1,4-cyclohexanediol diacrylate, 1,3-adamantanediol diacrylate, 1,3-adamantanedimethyl diacrylate, 2,2-diethyl-1,3-propanediol diacrylate, 2,2-diisobutyl-1,3-propanediol diacrylate, 1,3-cyclohexanedimethyl diacrylate, 1,4-cyclohexanedimethyl diacrylate; neopentyl glycol diacrylate, tetracthyleneglycol diacrylate, polyethyleneglycol diacrylate; and their corresponding methacrylates.

In a non-limiting illustrative embodiment, suitable crosslinking agents include, for example, 1,4-butanediol diacrylate, 1,4-butanediol dimethacrylate, ethylene glycol dimethacrylate, diethylene glycol diacrylate, diethylene glycol dimethacrylate, tricthylene glycol diacrylate, triethylene glycol dimethacrylate, tetracthylene glycol diacrylate, tetraethylene glycol dimethacrylate, 1,3-propanediol diacrylate, 1,3-propanediol dimethacrylate, 1,6-hexanediol diacrylate, 1,6-hexanediol dimethacrylate, poly(ethylene glycol) diacrylate (Mn=700 Daltons), poly(ethylene glycol) dimethacrylate (Mn=700 Daltons), and poly(ethylene glycol) dimethacrylate (Mn=1000 Daltons).

In non-limiting illustrative embodiments, as may be combined with one or more of the preceding paragraphs, the one or more crosslinking agents are present in the monomeric mixture in an ophthalmic device-forming amount. In non-limiting illustrative embodiments, as may be combined with one or more of the preceding paragraphs, the one or more crosslinking agents are present in the monomeric mixture in an amount of about 1 weight percent to about 10 weight percent, based on the total weight of the monomeric mixture. In non-limiting illustrative embodiments, as may be combined with one or more of the preceding paragraphs, the one or more crosslinking agents are present in the monomeric mixture in an amount of about 3 weight percent to about 5 weight percent, based on the total weight of the monomeric mixture.

In non-limiting illustrative embodiments, as may be combined with one or more of the preceding paragraphs, the monomeric mixture can further contain a reactive (polymerizable) ultraviolet (UV) absorber and/or a reactive blue-light absorber. Suitable reactive UV absorbers can be any known reactive UV absorber. In non-limiting illustrative embodiments, suitable reactive UV absorbers include, for example, 2-(2′-hydroxy-3′-methallyl-5′-methylphenyl)benzotriazole, commercially available as o-Methallyl Tinuvin P (“oMTP”) from Polysciences, Inc., Warrington, Pa., 3-(2H-benzo[d][1,2,3]triazol-2-yl)-4-hydroxyphenylethyl methacrylate, and 2-(3-(tert-butyl)-4-hydroxy-5-(5-methoxy-2H-benzo[d][1,2,3]triazol-2-yl)phenoxy)ethyl methacrylate.

In one illustrative embodiment, suitable reactive UV absorbers include, for example, one or more compounds of the following formulae:

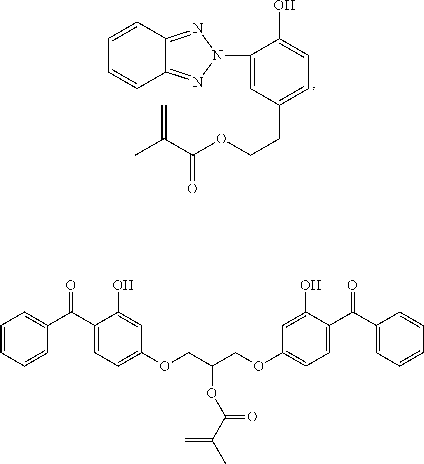 [SMILES: C=C(C)C(=O)OCCc1ccc(O)c(-n2nc3ccccc3n2)c1 ; C=C(C)C(=O)OC(COc1ccc(C(=O)c2ccccc2)c(O)c1)COc1ccc(C(=O)c2ccccc2)c(O)c1]

(2-Propenoic acid, 2-methyl,2-(4-benzoyl-3-hydroxyphenoxy)-1-[(4-benzoyl3-hydroxyphenoxy)methyl ester),

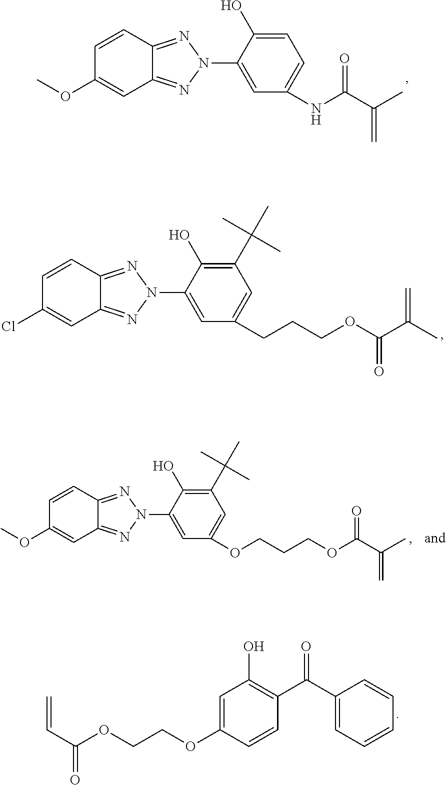

These compounds are merely illustrative and not intended to be limiting. Any known reactive UV absorber or later developed reactive UV absorber is contemplated for use herein.

In illustrative embodiments, the reactive UV absorbers can be present in the monomeric mixture in an amount ranging from about 0.1 weight percent to about 5 weight percent, based on the total weight of the monomeric mixture. In another illustrative embodiment, the reactive UV absorbers can be present in the monomeric mixture in an amount ranging from about 1.5 weight percent to about 2.5 weight percent, based on the total weight of the monomeric mixture. In yet another non-limiting illustrative embodiment, the reactive UV absorbers can be present in the monomeric mixture in an amount ranging from about 1.5 weight percent to about 2 weight percent, based on the total weight of the monomeric mixture.

Many reactive blue-light absorbing compounds are known. Preferred reactive blue-light absorbing compounds are those described in U.S. Pat. Nos. 5,470,932; 8,207,244; and 8,329,775, the contents of which are hereby incorporated by reference. In one embodiment, a blue-light absorbing dye is N-2-[3-(2′-methylphenylazo)-4-hydroxyphenyl]ethyl methacrylamide. In illustrative embodiments, the blue-light absorbers can be present in the monomeric mixture in an amount ranging from about 0.005 weight percent to about 1 weight percent, based on the total weight of the monomeric mixture. In another illustrative embodiment, the blue-light absorbers can be present in the monomeric mixture in an amount ranging from about 0.01 weight percent to about 1 weight percent, based on the total weight of the monomeric mixture.

In non-limiting illustrative embodiments, as may be combined with one or more of the preceding paragraphs, the monomeric mixture can contain one or more hydrophilic monomers. Suitable hydrophilic monomers include, for example, unsaturated carboxylic acids, acrylamides, vinyl lactams, hydroxyl-containing-(meth)acrylates, hydrophilic vinyl carbonates, hydrophilic vinyl carbamates, hydrophilic oxazolones, and poly(alkene glycols) functionalized with polymerizable groups and the like and mixtures thereof. Representative examples of unsaturated carboxylic acids include methacrylic acid, acrylic acid and the like and mixtures thereof. Representative examples of amides include alkylamides such as N,N-dimethylacrylamide, N,N-dimethylmethacrylamide and the like and mixtures thereof. Representative examples of cyclic lactams include N-vinyl-2-pyrrolidone, N-vinyl caprolactam, N-vinyl-2-piperidone and the like and mixtures thereof. Representative examples of hydroxyl-containing (meth)acrylates include 2-hydroxyethyl methacrylate (HEMA), glycerol methacrylate and the like and mixtures thereof. Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable hydrophilic monomers will be apparent to one skilled in the art. Mixtures of the foregoing hydrophilic monomers can also be used in the monomeric mixtures herein.

In an illustrative embodiment, the hydrophilic monomers are one or more of an acrylamide and a hydroxyl-containing-(meth)acrylate as defined above.

In a non-limiting illustrative embodiment, a hydrophilic monomer for use herein is a high glass transition temperature (Tg) hydrophilic monomer or homopolymer. A “high glass transition temperature hydrophilic monomer or homopolymer” is a hydrophilic monomer or homopolymer that, when incorporated into a polymer with one or more other monomers and cross-linkers, increases the glass transition temperature of the resulting polymer as compared to a resulting polymer formed without the high glass transition temperature hydrophilic monomer or homopolymer. In illustrative embodiments, the hydrophilic monomers and/or their corresponding homopolymer can have Tg values of greater than 50° C., e.g., Tg values between about 60 to about 100° C. For example, a hydrophilic monomer such as HEMA does not have a Tg value, however, when it is polymerized into its corresponding homopolymer (polyHEMA) then the corresponding homopolymer can have a Tg value of about 60° C. By raising the Tg value there is an added benefit of reducing surface tack which is advantageous for the fabrication of IOLs with minimal particulates and other cosmetic defects.

The ophthalmic devices of the illustrative embodiments described herein, e.g., intraocular lenses, can be prepared by polymerizing the foregoing monomeric mixtures to form a product that can be subsequently formed into the appropriate shape by, for example, lathing, injection molding, compression molding, cutting and the like. For example, the ophthalmic devices described herein can be prepared by combining the one or more cycloaliphatic (meth)acrylic monomers, one or more hydrophilic monomers; and one or more crosslinking agents and polymerizing the resulting mixture.

Polymerization may be facilitated by exposing the monomeric mixture to heat and/or radiation, such as ultraviolet light, visible light, or high energy radiation. A polymerization initiator may be included in the monomeric mixture to facilitate the polymerization step. Suitable polymerization initiators include thermal initiators and photoinitiators. Suitable free radical thermal polymerization initiators include, for example, organic peroxides such as acetyl peroxide, lauroyl peroxide, decanoyl peroxide, stearoyl peroxide, benzoyl peroxide, tertiarylbutyl peroxypivalate, peroxydicarbonate, and the like. Suitable free radical thermal polymerization initiators also include, for example, 2,2′-azobis(4-methoxy-2,4-dimethylvaleronitrile) (VAZO 33), 2,2′-Azobis[2-(2-imidazolin-2-yl)propane]dihydrochloride (VAZO 44), 2,2′-azobis(2-amidinopropane) dihydrochloride (VAZO 50), 2,2′-azobis(2,4-dimethylvaleronitrile) (VAZO 52), 2,2′-azobis(isobutyronitrile) (VAZO 64 or AIBN), 2,2′-azobis-2-methylbutyronitrile (VAZO 67), 1,1-azobis(1-cyclohexanecarbonitrile) (VAZO 88); 2,2′-azobis(2-cyclopropylpropionitrile), 2,2′-azobis(methylisobutyrate), 4,4′-Azobis(4-cyanovaleric acid), and combinations thereof. In one embodiment, a thermal initiator is 2,2′-azobis(isobutyronitrile) (VAZO 64 or AIBN).

Representative UV initiators are those known in the art and include benzoin methyl ether, benzoin ethyl ether, Darocure® 1173, 1164, 2273, 1116, 2959, 3331 (EM Industries) and Irgacure® 184, 651 and 819 (Ciba-Geigy), and the like.

In illustrative embodiments, the UV initiator can be employed in the monomeric mixture at a concentration of about 0.01 weight percent to about 5 weight percent, based on the total weight of the monomeric mixture. In other illustrative embodiments, the initiator can be employed in the monomeric mixture at a concentration of about 0.01 weight percent to about 3 weight percent, based on the total weight of the monomeric mixture. In other illustrative embodiments, the initiator can be employed in the monomeric mixture at a concentration of about 0.01 weight percent to about 1.5 weight percent, based on the total weight of the monomeric mixture.

Generally, polymerization can be carried out for about 15 minutes to about 72 hours, and under an inert atmosphere of, for example, nitrogen or argon. If thermoplastic molds made from various types of polymer resins are used, the molds can be treated with an inert gas, such as nitrogen or argon, prior to use. The resulting crude polymerization product can be extracted using an organic solvent, such as for example, acetone or isopropyl alcohol to remove unreacted components or side products which typically form during free radical polymerizations. If desired, the resulting polymerization product can be dried under vacuum, e.g., for about 5 to about 72 hours to remove residual solvent. The final product can be packaged as a dry lens or left in an aqueous solution in the final packaging configuration.

If necessary, it may be desirable to remove residual diluent from the lens before edge-finishing operations which can be accomplished by evaporation at or near ambient pressure or under vacuum. An elevated temperature can be employed to shorten the time necessary to evaporate the diluent. The time, temperature and pressure conditions for the solvent removal step will vary depending on such factors as the volatility of the diluent and the specific monomeric components, as can be readily determined by one skilled in the art. If desired, the mixture used to produce the hydrogel lens may further include crosslinking and wetting agents known in the prior art for making hydrogel materials.

The ophthalmic devices such as intraocular lenses obtained herein may be subjected to optional machining operations. For example, the optional machining steps may include buffing or polishing a lens edge and/or surface. Generally, such machining processes may be performed before or after the product is released from a mold part, e.g., the lens is dry released from the mold by employing vacuum tweezers to lift the lens from the mold, after which the lens is transferred by means of mechanical tweezers to a second set of vacuum tweezers and placed against a rotating surface to smooth the surface or edges. The lens may then be turned over in order to machine the other side of the lens.

The lens may then be transferred to individual lens packages containing a buffered saline solution. The saline solution may be added to the package either before or after transfer of the lens. Appropriate packaging designs and materials are known in the art. A plastic package is releasably sealed with a film. Suitable sealing films are known in the art and include foils, polymer films and mixtures thereof. The sealed packages containing the lenses are then sterilized to ensure a sterile product. Suitable sterilization means and conditions are known in the art and include, for example, autoclaving.

As one skilled in the art will readily appreciate other steps may be included in the molding and packaging process described above. Such other steps can include, for example, coating the formed lens, surface treating the lens during formation (e.g., via mold transfer), inspecting the lens, discarding defective lenses, cleaning the mold halves, reusing the mold halves, and the like and combinations thereof.

The ophthalmic devices described herein are intended for direct contact with body tissue or body fluid. As used herein, the term “ophthalmic device” refers to devices that reside in or on the eye. These devices can provide optical correction, wound care, drug delivery, diagnostic functionality or cosmetic enhancement or effect or a combination of these properties.

In illustrative embodiments, the ophthalmic device comprises a lens, inlay, outlay, or insert selected from an intraocular implant or lens, a contact lens, a corneal inlay, a corneal outlay, and a corneal insert.

In additional illustrative embodiments, the ophthalmic device is an intraocular implant or lens. Specifically, the ophthalmic device described herein includes intraocular implants and/or lenses made at least partially or completely from the polymerized monomeric mixtures described herein. Such intraocular implants or lenses can include an optic portion and one or more haptic portions. For example, the polymerized monomeric mixtures described herein will make up part or all of the optic portion of the intraocular implant or lens. In some embodiments, the optic portion of the implant or lens will have a core made from one of the polymerized monomeric mixtures described herein surrounded by a different polymer or material. Implants or lenses in which the optic portion is made up of, at least partially, one of the polymerized monomeric mixtures described herein will usually also have a haptic portion. The haptic portion can also be made of the polymerized monomeric mixtures described herein or can be made of a different material, for example another polymer.

In some embodiments, the intraocular implant or lens is a one-piece lens having a soft, foldable central optic region and an outer peripheral region (haptic-region) in which both regions are made of the same polymer. In other embodiments, the optic and haptic regions can be formed from different types of polymers or materials, if desired. Some implants or lenses can also have haptic portions that are made up of different materials, for example where one or more haptic portions is made from the same material as the optic portion and other haptic portions are made of materials other than the polymerized monomeric mixtures described herein. Multicomponent implants or lenses can be made by embedding one material in the other, concurrent extrusion processes, solidifying the hard material about the soft material, or forming an interpenetrating network of the rigid component into a preformed hydrophobic core. In instances where one or more haptic portions are made from a different material than the optic portion of the lens, the haptic portion can be attached to the optic portion in any manner known in the art, such as by drilling a hole or holes in the optic portion and inserting the haptic portion.

The polymerized monomeric mixtures described herein have been designed so that they are capable of being folded so that the intraocular lens can be inserted into the eye of an individual through a small incision. In illustrative embodiments, that incision will be less than about 2.5 mm. In other illustrative embodiments, the incision will be less than about 2 mm. The haptic portion of the lens provides the required support for the implant or lens in the eye after insertion and unfolding of the lens and tends to help stabilize the position of the lens after insertion and the closure of the incision. The shape of the haptic portion design is not particularly limited and can be any desired configuration, for example, either a plate type or graduated thickness spiral filaments, also known as a C-loop design.

The optic portion of the intraocular lens can be approximately 2 mm to about 6 mm in diameter prior to hydration. The about 2 to about 6 mm diameter is fairly standard in the art and is generally chosen to cover the pupil in its fully dilated state under naturally occurring conditions. However, this size is not limited to any particular diameter or size of intraocular lens, and other sizes are contemplated. Furthermore, it is not necessary that the lens optic portion be circular; it could also be oval, square, or any other shape as desired.

The intraocular lens can further include one or more non-optical haptic components extending away from the outermost peripheral surface of the optic portion. The haptic components can be of any desired shape, for example, graduated spiral filaments or flat plate sections and are used to support the lens within the posterior chamber of the eye. Lenses having any desired design configuration can be fabricated. Should the intraocular lens include other components besides the optical and haptic portions, such other portions can be made of a polymer as are the haptic and optic portions, or if desired, another material.

The intraocular implants lenses may be inserted into the eye in any manner known in the art. For example, in one embodiment, the intraocular lens may be folded prior to insertion into the eye using an intraocular lens inserter or by small, thin forceps of the type typically used by ophthalmic surgeons. After the implant or lens is in the targeted location, it is released to unfold. As is well known in the art, typically the lens that is to be replaced is removed prior to insertion of the intraocular lens. The intraocular lens described herein can be made of a generally physiologically inert soft polymeric material that is capable of providing a clear, transparent, refractive lens body even after folding and unfolding. In some embodiments, the foldable intraocular lens can be inserted into any eye by injection whereby the mechanically compliant material is folded and forced through a small tube such as a 1 mm to 3 mm inner diameter tube.

The following examples are provided to enable one skilled in the art to practice the invention and are merely illustrative. The examples should not be read as limiting the scope of the invention as defined in the claims.

Various polymerization products were formed as discussed below and characterized by standard testing procedures such as:

Water %: Two sets of six hydrated lenses or films are blotted dry on a piece of filter paper to remove excess water, and samples are weighed (wet weight). Samples are then placed in a microwave oven for 10 minutes inside a jar containing desiccant. The samples are then allowed to sit for 30 minutes to equilibrate to room temperature and reweighed (dry weight). The percent water is calculated from the wet and dry weights.

Refractive Index Test Method: Refractive index was measured using an Anton Paar Abbemat WR-wavelength refractometer. The instrument was equilibrated at either 25° C. or 35° C. for a minimum of 1 hour prior to use. The measurement wavelength was set at 589.3 nanometers. Using a pair of tweezers, the sample was placed on the quartz plate. The instrument lid was closed, and the refractive index was recorded after 60 seconds of dwell time. Measurements were performed on three polymer buttons, and the average was reported. In some examples, where it is noted, measurements were performed on both sides of the three polymer buttons, and the average of the six measurements was reported.

Abbe Number Test Method: Following the steps for measuring the refractive index at 589.3 nm, the refractive index at 486.1 nm and 656.3 nm were determined. Measurements were performed on three polymer buttons, and for each polymer button, the refractive index measurements at all three wavelengths were completed before measuring the next replicate. The Abbe number was calculated as follows:

$${V}_{D} = \frac{{n}_{D} - 1}{{n}_{F} - {n}_{C}}$$

where n_{D}, n_{F} and n_{C} are the refractive indices of the material at the wavelengths of the Fraunhofer D-, F- and C-spectral lines (589.3 nm, 486.1 nm and 656.3 nm, respectively). The average of the three measurements was reported. In some examples, where it is noted, measurements were performed on both sides of the three polymer buttons, and the average of the six measurements was reported.

Glass Transition Temperature Test Method: Because of the thickness and/or brittleness of the polymer buttons, test samples were cut from the center of the polymer buttons or lenses using a razor blade. The samples could not be punched out as with a thin film. Test samples were analyzed (in duplicate) on a DSC Q2000 TA instrument at heating rates of 10° C./minute and cooling rates of 5° C./minute under a nitrogen gas atmosphere.

The glass transition temperatures were determined either pre-extraction or post extraction.

In the examples, the following abbreviations are used.
PEA: 2-Phenylethyl Acrylate.
PEMA: 2-Phenylethyl Methacrylate.
PyPA: 3-(2-oxopyrrolidinyl)propyl Acrylate.
PyEMA: 3-(2-oxopyrrolidinyl)ethyl Methacrylate.
PyPMA: 3-(2-oxopyrrolidinyl)propyl Methacrylate.
BDDA: 1,4-Butanediol Diacrylate.
X-Monomer: 2-Methacrylic acid 3-[3-tert-butyl-5-(5-chlorobenzotriazol-2-yl)-4-hydroxyphenyl]-propyl ester.
EGDA: Ethylene Glycol Diacrylate.
AIBN: Azobisisobutyronitrile for thermal curing polymerization.
Irg 819: Irgacure® 819, a photoinitiator for free radical photo curing polymerization available from Sigma Aldrich.

### EXAMPLES 1-6 and COMPARATIVE EXAMPLE A

A monomeric mixture was made by mixing the following components, listed in Table 1 at amounts per weight percent, based on the total weight of the monomeric mixture.

TABLE 1

| | Comp. | | | | | | |
|---|---|---|---|---|---|---|---|
| | Ex. A | Ex. 1 | Ex. 2 | Ex. 3 | Ex. 4 | Ex. 5 | Ex. 6 |
| Formulation | | | | | | | |
| PEA | — | 44.98 | 54.92 | 64.51 | 74.15 | 44.47 | 30.62 |
| PEMA | — | — | — | — | — | 20.68 | 14.16 |
| PyPA | 94.46 | 49.62 | 39.55 | 30.03 | 20.15 | 29.57 | 49.85 |
| BDDA | 3.98 | 3.82 | 4.05 | 3.9 | 4.23 | 3.78 | 3.87 |
| X-Monomer | 1.04 | 1.00 | 1.00 | 1.00 | 0.98 | 1.01 | 1.00 |
| AIBN | 0.52 | 0.57 | 0.48 | 0.56 | 0.49 | −0 | — |
| IRG 819 | — | — | — | — | — | 0.49 | 0.49 |
| Properties | | | | | | | |
| Water content, % | 46.5 | 9.8 | 5.7 | 3.1 | 1.6 | 2.9 | 8.7 |
| RI (589 nm, 20° C.) | 1.424 | 1.521 | 1.534 | 1.541 | 1.547 | 1.545 | 1.523 |
| Tg (DSC, ° C.) | 7.2 | 7.7 | 7.7 | 7.5 | 6.6 | 12.6 | 12.5 |
| Abbe Number | 47 | 40 | 38 | 39 | 38 | 38 | 39 |

The monomeric mixtures were mold cast into intraocular contact lenses (+21.0 D or +34.0 D) and flat discs (˜1 cm diameter and 0.5 to 1.0 mm thickness). The formulation components were combined in a 20 mL scintillation vial, vortex mixed for 1 to 3 minutes, and syringe filtered through 1.0 μm and 0.2 μm AcroDisc PTFE syringe filters, respectively. Approximately 80 to 120 μL of the monomeric mixtures were dispensed and pressed against 2 polypropylene mold halves which were previously stored under nitrogen for at least 20 hours prior to use. Thermal cure cycle: the mold assemblies were placed in a pre-heated oven set at 110° C. The samples were heated for 20 minutes at 110° C. and 3 hours at 100° C. Photo cure cycle: The samples were cast in a yellow lab with suitable light filters and placed in a nitrogen filled glove box containing a fluorescent light fixture (UVP Ultraviolet Transilluminator, Type T-8, 15 Watts, Medium Bipin) and lamps (F15T8/BL/421 nm, LDC M2-7S-05 Hg). Samples were placed on a sheet of borosilicate glass and placed directly on top of the lamp assembly. The unit was turned on and samples were cured for 2 to 4 hours.

Lenses and discs were demolded and extracted in acetone for 20 hours at ambient temperature, air dried for a minimum of 5 hours, and then dried under vacuum (1 to 5 mm Hg) for at least 20 hours at 60 to 90° C. Samples were further hydrated in deionized water for at least 20 hours to calculate the equilibrium water content (EWC): EWC=100×(hydrated mass−solvent extracted dry mass)/hydrated mass. Refractive index and Abbe values were measured using a Schmidt+Haensch ATR critical angle refractometer at 20° C. RI measurements at 589 nm were verified using an Atago Multi-Wavelength Abbe Refractometer DR-M2.

### EXAMPLES 7-12

A monomeric mixture was made by mixing the following components, listed in Table 2 at amounts per weight percent, based on the total weight of the monomeric mixture.

TABLE 2

| | Ex. 7 | Ex. 8 | Ex. 9 | Ex. 10 | Ex. 11 | Ex. 12 |
|---|---|---|---|---|---|---|
| Formulation | | | | | | |
| PEA | 55.13 | 65.05 | 74.61 | 54.89 | 64.54 | 74.78 |
| PyEMA | 39.82 | 29.82 | 20.17 | 39.6 | 30.27 | 20.03 |
| BDDA | 3.77 | 3.87 | 3.90 | 4.00 | 3.67 | 3.70 |
| X-Monomer | 0.94 | 0.97 | 0.98 | 0.99 | 0.97 | 0.93 |
| AIBN | — | — | — | 0.53 | 0.55 | 0.57 |
| IRG 819 | 0.34 | 0.29 | 0.34 | — | — | — |
| Properties | | | | | | |
| Water content, % | 5.6 | 3.2 | 1.58 | 5.2 | 3.0 | 1.5 |
| RI (589 nm, 20° C.) | 1.534 | 1.538 | 1.545 | 1.531 | 1.543 | 1.545 |
| Tg (DSC, ° C.) | 27.3 | 21.3 | 15.2 | 30.0 | 22.8 | 15.6 |
| Abbe Number | 43 | 41 | 41 | 43 | 41 | 41 |

The monomeric mixtures were mold cast into intraocular contact lenses (+21.0 D or +34.0 D) and flat discs (˜1 cm diameter and 0.5 to 1.0 mm thickness). The formulation components were combined in a 20 mL scintillation vial, vortex mixed for 1 to 3 minutes, and syringe filtered through 1.0 μm and 0.2 μm AcroDisc PTFE syringe filters, respectively. Approximately 80 to 120 μL of the monomeric mixtures were dispensed and pressed against 2 polypropylene mold halves which were previously stored under nitrogen for at least 20 hours prior to use. Thermal cure cycle: the mold assemblies were placed in a pre-heated oven set at 110° C. The samples were heated for 20 minutes at 110° C. and 3 hours at 100° C. Photo cure cycle: The samples were cast in a yellow lab with suitable light filters and placed in a nitrogen filled glove box containing a fluorescent light fixture (UVP Ultraviolet Transilluminator, Type T-8, 15 Watts, Medium Bipin) and lamps (F15T8/BL/421 nm, LDC M2-7S-05 Hg). Samples were placed on a sheet of borosilicate glass and placed directly on top of the lamp assembly. The unit was turned on and samples were cured for 2 to 4 hours.

Lenses and discs were demolded and extracted in acetone for 20 hours at ambient temperature, air dried for a minimum of 5 hours, and then dried under vacuum (1 to 5 mm Hg) for at least 20 hours at 60 to 90° C. Samples were further hydrated in deionized water for 20 hours to calculate the equilibrium water content (EWC): EWC=100×(hydrated mass−solvent extracted dry mass)/hydrated mass. Refractive index and Abbe values were measured using a Schmidt+Haensch ATR critical angle refractometer at 20° C. RI measurements at 589 nm were verified using an Atago Multi-Wavelength Abbe Refractometer DR-M2.

### EXAMPLES 13-19

A monomeric mixture was made by mixing the following components, listed in Table 3 at amounts per weight percent, based on the total weight of the monomeric mixture.

TABLE 3

| | Ex. 13 | Ex. 14 | Ex. 15 | Ex. 16 | Ex. 17 | Ex. 18 | Ex. 19 |
|---|---|---|---|---|---|---|---|
| Formulation | | | | | | | |
| PEA | 44.84 | 54.84 | 54.72 | 59.80 | 64.70 | 74.78 | 79.80 |
| PyPMA | 49.88 | 40.09 | 39.89 | 35.10 | 29.81 | 20.07 | 15.01 |
| EGDMA | 3.99 | 3.76 | 3.99 | 3.83 | 4.18 | 3.89 | 3.90 |
| X-Monomer | 0.99 | 1.02 | 0.97 | 0.98 | 1.02 | 1.00 | 1.00 |
| IRG 819 | 0.30 | 0.28 | 0.43 | 0.29 | 0.28 | 0.25 | 0.29 |
| Properties | | | | | | | |
| Water content, % | 8.60 | 5.15 | 5.30 | 3.92 | 2.90 | 1.62 | 1.08 |
| RI (589 nm, 20° C.) | 1.511 | 1.530 | 1.529 | 1.534 | 1.537 | 1.545 | 1.548 |
| Tg (DSC, ° C.) | 25.5 | 20.2 | 21.5 | 18.6 | 17.0 | 12.6 | 10.9 |
| Abbe Number | 45 | 44 | 44 | 43 | 43 | 42 | 40 |

The monomeric mixtures were mold cast into intraocular contact lenses (+21.0 D or +34.0 D) and flat discs (˜1 cm diameter and 0.5 to 1.0 mm thickness). The formulation components were combined in a 20 mL scintillation vial, vortex mixed for 1 to 3 minutes, and syringe filtered through 1.0 μm and 0.2 μm AcroDisc PTFE syringe filters, respectively. Approximately 80 to 120 μL of the monomeric mixtures were dispensed and pressed against 2 polypropylene mold halves which were previously stored under nitrogen for at least 20 hours prior to use. Thermal cure cycle: the mold assemblies were placed in a pre-heated oven set at 110° C. The samples were heated for 20 minutes at 110° C. and 3 hours at 100° C. Photo cure cycle: The samples were cast in a yellow lab with suitable light filters and placed in a nitrogen filled glove box containing a fluorescent light fixture (UVP Ultraviolet Transilluminator, Type T-8, 15 Watts, Medium Bipin) and lamps (F15T8/BL/421 nm, LDC M2-7S-05 Hg). Samples were placed on a sheet of borosilicate glass and placed directly on top of the lamp assembly. The unit was turned on and samples were cured for 2 to 4 hours.

Lenses and discs were demolded and extracted in acetone for 20 hours at ambient temperature, air dried for a minimum of 5 hours, and then dried under vacuum (1 to 5 mm Hg) for at least 20 hours at 60 to 90° C. Samples were further hydrated in deionized water for 20 hours to calculate the equilibrium water content (EWC): EWC=100×(hydrated mass−solvent extracted dry mass)/hydrated mass. Refractive index and Abbe values were measured using a Schmidt+Haensch ATR critical angle refractometer at 20° C. RI measurements at 589 nm were verified using an Atago Multi-Wavelength Abbe Refractometer DR-M2.

In accordance with an aspect of the disclosure, am ophthalmic device is a polymerization product of a monomeric mixture comprising:
(a) one or more heterocyclic monomers having an ethylenically unsaturated reactive end group,
(b) one or more aromatic monomers having an ethylenically unsaturated reactive end group, and
(c) one or more crosslinking agents;
wherein the ophthalmic device has a refractive index of greater than or equal to about 1.50.

In non-limiting illustrative embodiments, as may be combined with one or more of the preceding paragraphs, the one or more heterocyclic monomers having an ethylenically unsaturated reactive end group comprise one or more monocyclic heterocyclic monomers having an ethylenically unsaturated reactive end group.

In non-limiting illustrative embodiments, as may be combined with one or more of the preceding paragraphs, the monocyclic heterocyclic monomers having an ethylenically unsaturated reactive end group comprises a C₃-C₈ heterocyclic monomers having an ethylenically unsaturated reactive end group.

In non-limiting illustrative embodiments, as may be combined with one or more of the preceding paragraphs, the one or more heterocyclic monomers having an ethylenically unsaturated reactive end group comprise one or more heterocyclic (meth)acrylic monomers.

In non-limiting illustrative embodiments, as may be combined with one or more of the preceding paragraphs, the (meth)acrylic group of the one or more heterocyclic (meth)acrylic monomers are (meth)acrylate-containing reactive end groups.

In non-limiting illustrative embodiments, as may be combined with one or more of the preceding paragraphs, the (meth)acrylate-containing reactive end group is represented by the structure:

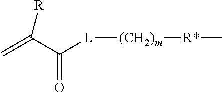

wherein R is hydrogen or methyl; L is O, NR¹, or S, where R¹ is H, CH₃, CH₂CH₃, or CH(CH₃)₂; m is an integer from 0 to 4 and R* is a linking group or bond.

In non-limiting illustrative embodiments, as may be combined with one or more of the preceding paragraphs, R* is a linking group selected from the group consisting of a straight or branched, substituted or unsubstituted C₁-C₆ alkyl group, and an —OR² group where R² is an alkyl group from 1 to 6 carbon atoms.

In non-limiting illustrative embodiments, as may be combined with one or more of the preceding paragraphs, L is O, m is from 1 to 4 and R* is a bond.

In non-limiting illustrative embodiments, as may be combined with one or more of the preceding paragraphs, the one or more heterocyclic monomers having an ethylenically unsaturated reactive end group comprise one or more heterocyclic (meth)acrylic monomers represented by a structure of Formula I:

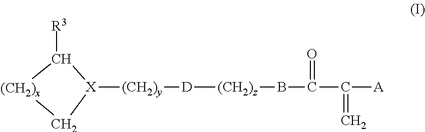

wherein x is an integer from 1 to 6, or from 1 to 5, or from 1 to 4 or from 1 to 2; y is an integer from 0 to 3 or from 1 to 3; R³ is hydrogen, hydroxy group, halogen group, carboxyl group, cyano group, nitro group, oxo (═O) group, thio (═S) group, a substituted or unsubstituted alkyl group, or a substituted or unsubstituted alkoxy group; X is a heteroatom such as O, N, S or P; B is O, NR, or S, where R is H, CH₃, CH₂CH₃, or CH(CH₃)₂; D is O, S, or a bond; A is H or CH₃; and z is 0 to 4, provided that if D is a bond then at least one of y and z is 0.

In non-limiting illustrative embodiments, as may be combined with one or more of the preceding paragraphs, x is an integer from 1 to 4; y is 0; R³ is an oxo group, X is N; D is O; z is an integer from 1 to 4; B is O; and A is H or CH₃.

In non-limiting illustrative embodiments, as may be combined with one or more of the preceding paragraphs, x is an integer from 1 to 3; y is 0; R³ is an oxo group, X is N; D is O; z is an integer from 2 to 3; B is O; and A is H or CH₃.

In non-limiting illustrative embodiments, as may be combined with one or more of the preceding paragraphs, x is an integer from 1 or 2; y is an integer from 1 to 4; R³ is an oxo group, X is N, D is a bond; z is 0; B is O; and A is H or CH₃.

In non-limiting illustrative embodiments, as may be combined with one or more of the preceding paragraphs, the one or more heterocyclic (meth)acrylic monomers are selected from the group consisting of 3-(2-oxopyrrolidin-1-yl)propyl acrylate, 2-(2-oxopyrrolidin-1-yl)ethyl acrylate, 3-(2-oxopyrrolidin-1-yl)propyl methacrylate, 2-(2-oxopyrrolidin-1-yl)ethyl methacrylate, (2-oxopyrrolidin-1-yl)methyl methacrylate, 4-(2-oxopyrrolidin-1-yl)butyl methacrylate, (2-oxopyrrolidin-1-yl)methyl acrylate, 4-(2-oxopyrrolidin-1-yl)butyl acrylate, 3-(1H-pyrrol-1-yl)propyl acrylate), 3-(1H-pyrrol-1-yl)propyl methacrylate and mixtures thereof.

In non-limiting illustrative embodiments, as may be combined with one or more of the preceding paragraphs, the aromatic group of the aromatic monomers having an ethylenically unsaturated reactive end group is a substituted or unsubstituted monoaromatic or polyaromatic radical containing from about 5 to about 25 carbon atoms.

In non-limiting illustrative embodiments, as may be combined with one or more of the preceding paragraphs, the aromatic group of the aromatic monomers having an ethylenically unsaturated reactive end group is a substituted or unsubstituted monoaromatic radical containing from 6 to 12 carbon atoms directly bonded to an alkyl group.

In non-limiting illustrative embodiments, as may be combined with one or more of the preceding paragraphs, the one or more aromatic monomers having an ethylenically unsaturated reactive end group comprise one or more aromatic (meth)acrylic monomers.

In non-limiting illustrative embodiments, as may be combined with one or more of the preceding paragraphs, the (meth)acrylic group of the one or more aromatic (meth)acrylic monomers are (meth)acrylate-containing reactive end groups.

In non-limiting illustrative embodiments, as may be combined with one or more of the preceding paragraphs, the (meth)acrylate-containing reactive end group is represented by the structure:

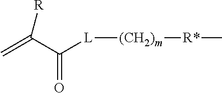

wherein R is hydrogen or methyl; L is O, NR¹, or S, where R¹ is H, CH₃, CH₂CH₃, or CH(CH₃)₂; m is an integer from 0 to 4 and R* is a linking group or bond.

In non-limiting illustrative embodiments, as may be combined with one or more of the preceding paragraphs, R* is a linking group selected from the group consisting of a straight or branched, substituted or unsubstituted C₁-C₆ alkyl group, and an —OR² group where R² is an alkyl group from 1 to 6 carbon atoms.

In non-limiting illustrative embodiments, as may be combined with one or more of the preceding paragraphs, L is O, m is from 1 to 4 and R* is a bond.

In non-limiting illustrative embodiments, as may be combined with one or more of the preceding paragraphs, the aromatic (meth)acrylic monomer is selected from the group consisting of 2-phenylethyl acrylate, 2-phenylethyl methacrylate 2-phenoxyethyl acrylate, 2-phenoxyethyl methacrylate, 3-phenylpropyl acrylate, 3-phenylpropyl methacrylate and mixtures thereof

In non-limiting illustrative embodiments, as may be combined with one or more of the preceding paragraphs, the monomeric mixture comprises:
(a) about 20 weight percent to about 75 weight percent, based on the total weight of the monomeric mixture, of the one or more heterocyclic monomers having an ethylenically unsaturated reactive end group,
(b) about 30 weight percent to about 80 weight percent, based on the total weight of the monomeric mixture, of the one or more aromatic monomers having an ethylenically unsaturated reactive end group, and
(c) about 1 weight percent to about 10 weight percent, based on the total weight of the monomeric mixture, of the one or more crosslinking agents.

In non-limiting illustrative embodiments, as may be combined with one or more of the preceding paragraphs, the monomeric mixture comprises:
(a) about 20 weight percent to about 50 weight percent, based on the total weight of the monomeric mixture, of the one or more heterocyclic monomers having an ethylenically unsaturated reactive end group,
(b) about 40 weight percent to about 80 weight percent, based on the total weight of the monomeric mixture, of the one or more aromatic monomers having an ethylenically unsaturated reactive end group, and
(c) about 3 weight percent to about 5 weight percent, based on the total weight of the monomeric mixture, of the one or more crosslinking agents.

In non-limiting illustrative embodiments, as may be combined with one or more of the preceding paragraphs, the one or more crosslinking agents are selected from the group consisting of 1,4-butanediol diacrylate, 1,4-butanediol dimethacrylate, ethylene glycol dimethacrylate, diethylene glycol) diacrylate, diethylene glycol) dimethacrylate, triethylene glycol diacrylate, triethylene glycol dimethacrylate, tetraethylene glycol diacrylate, tetraethylene glycol dimethacrylate, 1,3-propanediol diacrylate, 1,3-propanediol dimethacrylate, 1,6-hexanediol diacrylate, 1,6-hexanediol dimethacrylate, poly(ethylene glycol) diacrylate, poly(ethylene glycol) dimethacrylate, and poly(ethylene glycol) dimethacrylate.

In non-limiting illustrative embodiments, as may be combined with one or more of the preceding paragraphs, the monomeric mixture further comprises one or more of a reactive ultraviolet absorber and a reactive blue-light absorber.

In non-limiting illustrative embodiments, as may be combined with one or more of the preceding paragraphs, the monomeric mixture comprises from about 0.1 weight percent to about 5 weight percent, based on the total weight of the monomeric mixture, of the reactive ultraviolet absorber.

In non-limiting illustrative embodiments, as may be combined with one or more of the preceding paragraphs, the monomeric mixture further comprises one or more C₃ to C₁₂ aliphatic (meth)acrylate-containing monomers.

In non-limiting illustrative embodiments, as may be combined with one or more of the preceding paragraphs, the ophthalmic device has a Tg of less than 30° C. in the dry state.

In non-limiting illustrative embodiments, as may be combined with one or more of the preceding paragraphs, the ophthalmic device has an equilibrium water content (EWC) of greater than about 1% and up to about 10%.

In non-limiting illustrative embodiments, as may be combined with one or more of the preceding paragraphs, the ophthalmic device is a thermally cured polymerization product.

In non-limiting illustrative embodiments, as may be combined with one or more of the preceding paragraphs, the ophthalmic device is an intraocular lens.

In accordance with another aspect of the disclosure, a method for making an ophthalmic device, comprises:
(a) providing a monomeric mixture comprising:
(i) one or more heterocyclic monomers having an ethylenically unsaturated reactive end group,
(ii) one or more aromatic monomers having an ethylenically unsaturated reactive end group, and
(iii) one or more crosslinking agents, and
(b) subjecting the monomeric mixture to polymerization conditions to provide an ophthalmic device having a refractive index of greater than or equal to about 1.50.

In non-limiting illustrative embodiments, as may be combined with one or more of the preceding paragraphs, the one or more heterocyclic monomers having an ethylenically unsaturated reactive end group comprise one or more monocyclic heterocyclic monomers having an ethylenically unsaturated reactive end group.

In non-limiting illustrative embodiments, as may be combined with one or more of the preceding paragraphs, the monocyclic heterocyclic monomers having an ethylenically unsaturated reactive end group comprises a C₃-C₈ heterocyclic monomers having an ethylenically unsaturated reactive end group.

In non-limiting illustrative embodiments, as may be combined with one or more of the preceding paragraphs, the one or more heterocyclic monomers having an ethylenically unsaturated reactive end group comprise one or more heterocyclic (meth)acrylic monomers.

In non-limiting illustrative embodiments, as may be combined with one or more of the preceding paragraphs, the (meth)acrylic group of the one or more heterocyclic (meth)acrylic monomers are (meth)acrylate-containing reactive end groups.

In non-limiting illustrative embodiments, as may be combined with one or more of the preceding paragraphs, the (meth)acrylate-containing reactive end group is represented by the structure:

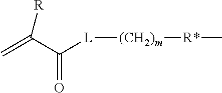

wherein R is hydrogen or methyl; L is O, NR¹, or S, where R¹ is H, CH₃, CH₂CH₃, or CH(CH₃)₂; m is an integer from 0 to 4 and R* is a linking group or bond.

In non-limiting illustrative embodiments, as may be combined with one or more of the preceding paragraphs, R* is a linking group selected from the group consisting of a straight or branched, substituted or unsubstituted C₁-C₆ alkyl group, and an —OR² group where R² is an alkyl group from 1 to 6 carbon atoms.

In non-limiting illustrative embodiments, as may be combined with one or more of the preceding paragraphs, L is O, m is from 1 to 4 and R* is a bond.

In non-limiting illustrative embodiments, as may be combined with one or more of the preceding paragraphs, the one or more heterocyclic monomers having an ethylenically unsaturated reactive end group comprise one or more heterocyclic (meth)acrylic monomers represented by a structure of Formula I:

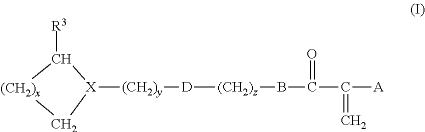

wherein x is an integer from 1 to 6, or from 1 to 5, or from 1 to 4 or from 1 to 2; y is an integer from 0 to 3 or from 1 to 3; R³ is hydrogen, hydroxy group, halogen group, carboxyl group, cyano group, nitro group, oxo (═O) group, thio (═S) group, a substituted or unsubstituted alkyl group, or a substituted or unsubstituted alkoxy group; X is a heteroatom such as O, N, S or P; B is O, NR, or S, where R is H, CH₃, CH₂CH₃, or CH(CH₃)₂; D is O, S, or a bond; A is H or CH₃; and z is 0 to 4, provided that if D is a bond then at least one of y and z is 0.

In non-limiting illustrative embodiments, as may be combined with one or more of the preceding paragraphs, x is an integer from 1 to 4; y is 0; R³ is an oxo group, X is N; D is O; z is an integer from 1 to 4; B is O; and A is H or CH₃.

In non-limiting illustrative embodiments, as may be combined with one or more of the preceding paragraphs, x is an integer from 1 to 3; y is 0; R³ is an oxo group, X is N; D is O; z is an integer from 2 to 3; B is O; and A is H or CH₃.

In non-limiting illustrative embodiments, as may be combined with one or more of the preceding paragraphs, x is an integer from 1 or 2; y is an integer from 1 to 4; R³ is an oxo group, X is N, D is a bond; z is 0; B is O; and A is H or CH₃.

In non-limiting illustrative embodiments, as may be combined with one or more of the preceding paragraphs, the one or more heterocyclic (meth)acrylic monomers are selected from the group consisting of 3-(2-oxopyrrolidin-1-yl)propyl acrylate, 2-(2-oxopyrrolidin-1-yl)ethyl acrylate, 3-(2-oxopyrrolidin-1-yl)propyl methacrylate, 2-(2-oxopyrrolidin-1-yl)ethyl methacrylate, (2-oxopyrrolidin-1-yl)methyl methacrylate, 4-(2-oxopyrrolidin-1-yl)butyl methacrylate, (2-oxopyrrolidin-1-yl)methyl acrylate, 4-(2-oxopyrrolidin-1-yl)butyl acrylate, 3-(1H-pyrrol-1-yl)propyl acrylate), 3-(1H-pyrrol-1-yl)propyl methacrylate and mixtures thereof.

In non-limiting illustrative embodiments, as may be combined with one or more of the preceding paragraphs, the aromatic group of the aromatic monomers having an ethylenically unsaturated reactive end group is a substituted or unsubstituted monoaromatic or polyaromatic radical containing from about 5 to about 25 carbon atoms.

In non-limiting illustrative embodiments, as may be combined with one or more of the preceding paragraphs, the aromatic group of the aromatic monomers having an ethylenically unsaturated reactive end group is a substituted or unsubstituted monoaromatic radical containing from 6 to 12 carbon atoms directly bonded to an alkyl group.

In non-limiting illustrative embodiments, as may be combined with one or more of the preceding paragraphs, the one or more aromatic monomers having an ethylenically unsaturated reactive end group comprise one or more aromatic (meth)acrylic monomers.

In non-limiting illustrative embodiments, as may be combined with one or more of the preceding paragraphs, the (meth)acrylic group of the one or more aromatic (meth)acrylic monomers are (meth)acrylate-containing reactive end groups.

In non-limiting illustrative embodiments, as may be combined with one or more of the preceding paragraphs, the (meth)acrylate-containing reactive end group is represented by the structure:

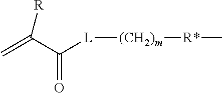

wherein R is hydrogen or methyl; L is O, NR¹, or S, where R¹ is H, CH₃, CH₂CH₃, or CH(CH₃)₂; m is an integer from 0 to 4 and R* is a linking group or bond.

In non-limiting illustrative embodiments, as may be combined with one or more of the preceding paragraphs, R* is a linking group selected from the group consisting of a straight or branched, substituted or unsubstituted C₁-C₆ alkyl group, and an —OR² group where R² is an alkyl group from 1 to 6 carbon atoms.

In non-limiting illustrative embodiments, as may be combined with one or more of the preceding paragraphs, L is O, m is from 1 to 4 and R* is a bond.

In non-limiting illustrative embodiments, as may be combined with one or more of the preceding paragraphs, the aromatic (meth)acrylic monomer is selected from the group consisting of 2-phenylethyl acrylate, 2-phenylethyl methacrylate 2-phenoxyethyl acrylate, 2-phenoxyethyl methacrylate, 3-phenylpropyl acrylate, 3-phenylpropyl methacrylate and mixtures thereof

In non-limiting illustrative embodiments, as may be combined with one or more of the preceding paragraphs, the monomeric mixture comprises:
(i) about 20 weight percent to about 75 weight percent, based on the total weight of the monomeric mixture, of the one or more heterocyclic monomers having an ethylenically unsaturated reactive end group,
(ii) about 30 weight percent to about 80 weight percent, based on the total weight of the monomeric mixture, of the one or more aromatic monomers having an ethylenically unsaturated reactive end group, and
(iii) about 1 weight percent to about 10 weight percent, based on the total weight of the monomeric mixture, of the one or more crosslinking agents.

In non-limiting illustrative embodiments, as may be combined with one or more of the preceding paragraphs, the monomeric mixture comprises:
(i) about 20 weight percent to about 50 weight percent, based on the total weight of the monomeric mixture, of the one or more heterocyclic monomers having an ethylenically unsaturated reactive end group,
(ii) about 40 weight percent to about 80 weight percent, based on the total weight of the monomeric mixture, of the one or more aromatic monomers having an ethylenically unsaturated reactive end group, and
(iii) about 3 weight percent to about 5 weight percent, based on the total weight of the monomeric mixture, of the one or more crosslinking agents.

In non-limiting illustrative embodiments, as may be combined with one or more of the preceding paragraphs, the one or more crosslinking agents are selected from the group consisting of 1,4-butanediol diacrylate, 1,4-butanediol dimethacrylate, ethylene glycol dimethacrylate, diethylene glycol) diacrylate, diethylene glycol) dimethacrylate, triethylene glycol diacrylate, triethylene glycol dimethacrylate, tetraethylene glycol diacrylate, tetraethylene glycol dimethacrylate, 1,3-propanediol diacrylate, 1,3-propanediol dimethacrylate, 1,6-hexanediol diacrylate, 1,6-hexanediol dimethacrylate, poly(ethylene glycol) diacrylate, poly(ethylene glycol) dimethacrylate, and poly(ethylene glycol) dimethacrylate.

In non-limiting illustrative embodiments, as may be combined with one or more of the preceding paragraphs, the monomeric mixture further comprises one or more of a reactive ultraviolet absorber and a reactive blue-light absorber.

In non-limiting illustrative embodiments, as may be combined with one or more of the preceding paragraphs, the monomeric mixture comprises from about 0.1 weight percent to about 5 weight percent, based on the total weight of the monomeric mixture, of the reactive ultraviolet absorber.

In non-limiting illustrative embodiments, as may be combined with one or more of the preceding paragraphs, the monomeric mixture further comprises one or more C₃ to C₁₂ aliphatic (meth)acrylate-containing monomers.

In non-limiting illustrative embodiments, as may be combined with one or more of the preceding paragraphs, the ophthalmic device has a Tg of less than 30° C. in the dry state.

In non-limiting illustrative embodiments, as may be combined with one or more of the preceding paragraphs, the ophthalmic device has an equilibrium water content (EWC) of greater than 2% and up to 9%.

In non-limiting illustrative embodiments, as may be combined with one or more of the preceding paragraphs, the step of subjecting the monomeric mixture to polymerizing conditions comprises subjecting the monomeric mixture to thermally curing polymerization conditions.

In non-limiting illustrative embodiments, as may be combined with one or more of the preceding paragraphs, the ophthalmic device is an intraocular lens.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present compositions and are disclosed herein just as if each and every such sub-combination was individually and explicitly disclosed herein.

It will be understood that various modifications may be made to the embodiments disclosed herein. Therefore, the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. An ophthalmic device which is a polymerization product of a monomeric mixture comprising:
(a) one or more heterocyclic monomers having an ethylenically unsaturated reactive end group;
(b) one or more aromatic monomers having an ethylenically unsaturated reactive end group; and
(c) one or more crosslinking agents;
wherein the ophthalmic device has a refractive index of greater than or equal to about 1.50.

2. The ophthalmic device according to claim 1, wherein the one or more heterocyclic monomers having an ethylenically unsaturated reactive end group comprise one or more monocyclic heterocyclic monomers having an ethylenically unsaturated reactive end group.

3. The ophthalmic device according to claim 2, wherein the monocyclic heterocyclic monomers having an ethylenically unsaturated reactive end group comprises a C₃-C₈ heterocyclic monomers having an ethylenically unsaturated reactive end group.

4. The ophthalmic device according to claim 1, wherein the one or more heterocyclic monomers having an ethylenically unsaturated reactive end group comprise one or more heterocyclic (meth)acrylic monomers.

5. The ophthalmic device according to claim 1, wherein the one or more heterocyclic monomers having an ethylenically unsaturated reactive end group comprise one or more heterocyclic (meth)acrylic monomers represented by a structure of Formula I: 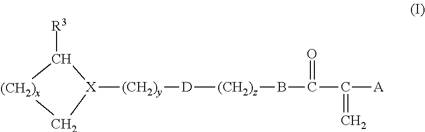
wherein x is an integer from 1 to 6; y is an integer from 0 to 3; R³ is hydrogen, a hydroxy group, a halogen group, a carboxyl group, a cyano group, a nitro group, an oxo (═O) group, a thio (═S) group, a substituted or unsubstituted alkyl group, or a substituted or unsubstituted alkoxy group; X is a heteroatom; D is O, S, or a bond; A is H or CH₃; and z is 0 to 4, provided that if D is a bond then at least one of y and z is 0.

6. The ophthalmic device according to claim 1, wherein the one or more heterocyclic (meth)acrylic monomers are selected from the group consisting of 3-(2-oxopyrrolidin-1-yl)propyl acrylate, 2-(2-oxopyrrolidin-1-yl)ethyl acrylate, 3-(2-oxopyrrolidin-1-yl)propyl methacrylate, 2-(2-oxopyrrolidin-1-yl)ethyl methacrylate, (2-oxopyrrolidin-1-yl)methyl methacrylate, 4-(2-oxopyrrolidin-1-yl)butyl methacrylate, (2-oxopyrrolidin-1-yl)methyl acrylate, 4-(2-oxopyrrolidin-1-yl)butyl acrylate, 3-(1H-pyrrol-1-yl)propyl acrylate), 3-(1H-pyrrol-1-yl)propyl methacrylate and mixtures thereof.

7. The ophthalmic device according to claim 1, wherein the aromatic group of the one or more aromatic monomers having an ethylenically unsaturated reactive end group is a substituted or unsubstituted monoaromatic or polyaromatic radical containing from about 5 to about 25 carbon atoms.

8. The ophthalmic device according to claim 1, wherein the aromatic group of the one or more aromatic monomers having an ethylenically unsaturated reactive end group is a substituted or unsubstituted monoaromatic radical containing from 6 to 12 carbon atoms directly bonded to an alkyl group.

9. The ophthalmic device according to claim 1, wherein the one or more aromatic monomers having an ethylenically unsaturated reactive end group comprise one or more aromatic (meth)acrylic monomers.

10. The ophthalmic device according to claim 1, wherein the one or more aromatic monomers having an ethylenically unsaturated reactive end group are one or more aromatic (meth)acrylic monomers selected from the group consisting of 2-phenylethyl acrylate, 2-phenylethyl methacrylate 2-phenoxyethyl acrylate, 2-phenoxyethyl methacrylate, 3-phenylpropyl acrylate, 3-phenylpropyl methacrylate and mixtures thereof.

11. The ophthalmic device according to 1, comprising:
(a) about 20 weight percent to about 75 weight percent, based on the total weight of the monomeric mixture, of the one or more heterocyclic monomers having an ethylenically unsaturated reactive end group;
(b) about 30 weight percent to about 80 weight percent, based on the total weight of the monomeric mixture, of the one or more aromatic monomers having an ethylenically unsaturated reactive end group; and
(c) about 1 weight percent to about 10 weight percent, based on the total weight of the monomeric mixture, of the one or more crosslinking agents.

12. The ophthalmic device according to claim 1, comprising:
(a) about 20 weight percent to about 50 weight percent, based on the total weight of the monomeric mixture, of the one or more heterocyclic monomers having an ethylenically unsaturated reactive end group;
(b) about 40 weight percent to about 80 weight percent, based on the total weight of the monomeric mixture, of the one or more aromatic monomers having an ethylenically unsaturated reactive end group; and
(c) about 3 weight percent to about 5 weight percent, based on the total weight of the monomeric mixture, of the one or more crosslinking agents.

13. The ophthalmic device according to claim 1, wherein the one or more crosslinking agents are selected from the group consisting of 1,4-butanediol diacrylate, 1,4-butanediol dimethacrylate, ethylene glycol dimethacrylate, diethylene glycol) diacrylate, diethylene glycol) dimethacrylate, triethylene glycol diacrylate, triethylene glycol dimethacrylate, tetraethylene glycol diacrylate, tetraethylene glycol dimethacrylate, 1,3-propanediol diacrylate, 1,3-propanediol dimethacrylate, 1,6-hexanediol diacrylate, 1,6-hexanediol dimethacrylate, poly(ethylene glycol) diacrylate, poly(ethylene glycol) dimethacrylate, and poly(ethylene glycol) dimethacrylate.

14. The ophthalmic device according to claim 1, wherein the monomeric mixture further comprises one or more of a reactive ultraviolet absorber and a reactive blue-light absorber.

15. The ophthalmic device according to claim 1, having a Tg of less than 30° C. in the dry state.

16. The ophthalmic device according to claim 1, having an equilibrium water content (EWC) of greater than about 1% and up to about 10%.

17. The ophthalmic device according to claim 1, which is a thermally cured polymerization product.

18. The ophthalmic device according to claim 1, which is an intraocular lens.

19. A method for making an ophthalmic device, comprising:
(a) providing a monomeric mixture comprising:
(i) one or more heterocyclic monomers having an ethylenically unsaturated reactive end group;
(ii) one or more aromatic monomers having an ethylenically unsaturated reactive end group; and
(iii) one or more crosslinking agents; and
(b) subjecting the monomeric mixture to polymerization conditions to provide an ophthalmic device having a refractive index of greater than or equal to about 1.50.

20. The method according to claim 19, wherein the monomeric mixture comprises:
(i) about 20 weight percent to about 75 weight percent, based on the total weight of the monomeric mixture, of the one or more heterocyclic monomers having an ethylenically unsaturated reactive end group;
(ii) about 30 weight percent to about 80 weight percent, based on the total weight of the monomeric mixture, of the one or more aromatic monomers having an ethylenically unsaturated reactive end group; and
(iii) about 1 weight percent to about 10 weight percent, based on the total weight of the monomeric mixture, of the one or more crosslinking agents.
